(19) United States
(12) Patent Application Publication
(10) Pub. No.: US 2010/0152849 A1
(43) Pub. Date: Jun. 17, 2010

(54) RETINAL PROSTHETIC DEVICES

(75) Inventors: Patrick Degenaar, London (GB); Mark Hankins, Oxford (GB); Emannuel Drakakis, London (GB); Christofer Toumazou, London (GB); Konstantin Nikolic, London (GB); Christopher Kennard, London (GB)

Correspondence Address: FRASER CLEMENS MARTIN & MILLER LLC, 28366 KENSINGTON LANE, PERRYSBURG, OH, 43551, US

(73) Assignee: IMPERIAL INNOVATIONS LIMITED, London (GB)

(21) Appl. No.: 12/306,072

(22) PCT Filed: Jun. 1, 2007
(86) PCT No.: PCT/GB2007/002019

§ 371 Date: Feb. 12, 2010

(30) Foreign Application Priority Data
Jun. 21, 2006 (GB) 0612242.8

(51) Int. Cl. A61F 2/14; A61F 9/00

(52) U.S. Cl. 623/6.63

(57) ABSTRACT

A retinal prosthetic device comprises image capture means (20) arranged to capture an image, light producing means (30) arranged to define a plurality of light paths along each of which a light beam can be directed towards a respective position on a retina, and control means (24) arranged to process the captured image and control the light producing means so as to produce a stimulating array of light beams along a group of the light paths, the group being dependent upon the captured image.

### FIELD OF THE INVENTION

The present invention relates to retinal prosthetic devices, and to the use of such devices to overcome partial or complete loss of vision.

### BACKGROUND TO THE INVENTION

About 0.8% of Americans and Europeans older than 40 years are blind according to the US definition of blindness (<20/200 best corrected visual acuity in the better-seeing eye). About 0.04% of the population suffer low vision and blindness due to retinal dystrophies classed collectively as retinitis pigmentosa. A further 1-2 percent of over 50′s and 25% of over 80's suffer from reduced vision due to age related macular degeneration (AMD). AMD increases dramatically with age, so that (with about 2 million cases in the USA) is the leading cause of blindness among Americans of European descent. These diseases result in the loss of photosensitivity primarily due to a lesion of the rod and cone photoreceptors, and medical intervention to date has been disappointing. There is no known mechanism by which the eye can self-repair. There are no drugs which can significantly slow the onset of most of these diseases, and none capable of restoring lost vision. Stem cell therapies are complex and may be many decades away from potential treatment. Prosthetic implants are therefore the only method at present by which we can offer a return of some of the lost vision.

There have been numerous attempts to implement retinal prostheses as a treatment strategy. These have usually been silicon chips which actively replace the non-functioning light sensing structures in the retina (rods and cones). The implants to date have come in two forms: sub-retinal and epi-retinal. Sub-retinal implants are placed underneath the retinal layers and usually consist of a micro-photodiode array which attempts to stimulate the remaining signal processing layers in the retina. Epi-retinal chips are positioned on the surface of the retina, and try to stimulate the retinal ganglion cell (RGC) layer. In this case, additional image processing is required to replicate that of the bypassed retinal layers. In most forms of retinal dystrophy the photoreceptors are lost, but the RGCs, the output neurones of the retinal network, survive and project to the retinorecipient areas of the visual brain (via the lateral geniculate nucleus to the primary visual cortex).

It is also possible to bypass the entire eye, and implant a grid of electrodes directly onto the visual cortex of the brain. A further possibility is to stimulate the optic nerve. Both of these approaches are much more invasive. There is also a much higher risk of problems due to infection or epileptic seizure. As with epi-retinal implants, image processing is required in addition to imaging, sensing and stimulation. Nevertheless, despite their invasive nature, these forms of implants are being actively researched.

Retinal prostheses represent a realistic near-term possibility for individuals with incurable retinal dystrophies. However, the rigid silicon structures that have been implanted to date have their own problems. In the case of sub-retinal implants the impermeable silicon structure can hasten the degradation of remaining retinal processing layers. The curved nature of the eye makes it very difficult to cover any significant portion of the retina, though there have been attempts at more flexible substrates. Furthermore power consumption issues are highly significant to these devices. Introduction of power cables is difficult, and power transmission through an RF link for example does not always provide sufficient power to power a significant quantity of stimulation points. Furthermore the implant contains invasive electrodes used to inject current into ganglion cells, which can cause further degradation of remaining functional tissue.

The present invention therefore relates to a prosthetic device based on optical stimulation rather than the use of electrodes. Some embodiments of the present invention modify neural cells to be photoactive, and some include methods of sensing the light, processing the signals to mimic the functioning of the retinal processing layers, and stimulating the ganglion cells using light emitting diodes.

Attempts to implement retinal implants to date are generally based on silicon chips which actively replace the non-functioning pigment epithelial light sensing structures in the retina. The implants to date have come in two forms: sub-retinal and epi-retinal. Both have to be implanted inside the eyeball (and sub-retinal structures need to be placed under the retina, requiring more delicate surgery) and the stimulating electrodes have to be in good physical contact with the cells which they stimulate. The epi-retinal implants stimulate the ganglion cells.

One known solution for ganglion cell stimulation is the use of an array of hard electrodes (gold, silver, platinum, iridium oxide, etc). The microelectrodes are usually photolithographically produced on rigid silicon substrates. Silicon substrates can be processed onto flexible structures, but they are fragile. Polymer-based electrode support structures might give the needed flexibility and some initial steps in that direction have been recently taken. These electrodes are used to directly inject current into the neural cells and trigger action potentials. However, this technique is invasive, can damage the cells and hasten long term degradation processes of remaining retinal tissue. Additionally, the position of the electrodes is fixed once they are inserted, and precise positioning on the micrometer scale is hard to control. It can therefore be a matter of chance whether individual electrodes are optimally placed or not.

Accordingly the present invention uses an optical method, and in some embodiments light is used for stimulation of neurons. Optical excitation allows for the energy source of the stimulation to be externalised and thus reduces the power constraints on the system. In addition, through optics, it is possible to stimulate over the whole retina rather than a small area, generally in the periphery, when solid state implants are used. It is known that light sensitized mammalian retinal ganglion cells can pass bursting electrical representations of the visual scene towards the visual cortex as shown by Bi et al, Bi A, Cui J, Ma Y-P, Olshevskaya E, Pu M, Dizhoor A M and Pan Z-H: “Ectopic Expression of a Microbial-Type Rhodopsin Restores Visual Responses in Mice with Photoreceptor Degeneration”, Neuron 50, 23-33 (2006). Humayun's group proposed the in vitro addition of Photosystem I (PSI)-proteoliposomes (a substance extracted from plants) to retinoblastoma cells. These methods are described by Greenbaum E, Humayun M, Kuritz T, Lee J W, Sanders C A, Bruce B and Lee I: “Nanoscale photosynthesis, the photophysics of neural cells and artificial sight”, Proceedings of the IEEE-EMBS Special Topic Conference on Molecular, Cellular and Tissue Engineering, 2002, Publication Date: 6-9 Jun. 2002 On page(s): 83-85, and by Kuritz, T.; Lee, I.; Owens, E. T.; Humayun, M.; Greenbaum, E. “Molecular photovoltaics and the photoactivation of mammalian cells”, IEEE Trans. Nanobioscience (2): 196-200 June 2005. They achieved some limited success, but had no proposal to take this work beyond in-vitro. In 2005 Hankins et al first described (Melyan Z, Tarttelin E E, Bellingham J, Lucas R J and Hankins M W, “Addition of human melanopsin renders mammalian cells photoresponsive” Nature vol. 433 pp. 741-745 (2005)) a method which might be developed into a clinical method. In this work genetic engineering was used to engender light sensitivity. Transfection methods were proposed to deliver the genetic change. Finally, Bi et al have demonstrated in vivo that it is possible to achieve the light sensitisation of (rodent) ganglion cells by using channelrhodopsin-2. They have also confirmed that the light-generated activities of the retinal ganglion cells can be transmitted to the visual cortex.

There has however, been no realistic system proposed that could be used as a practical prosthetic device based upon optical stimulation.

Accordingly the present invention provides a retinal prosthetic device comprising image capture means arranged to capture an image, light producing means arranged to define a plurality of light paths along each of which a light beam can be directed towards a respective position on a retina, and control means arranged to process the captured image and control the light producing means so as to produce a stimulating array of light beams along a group of the light paths, the group being dependent upon the captured image.

Some embodiments of the present invention are designed around the light sensitization of retinal ganglion cells, but modifications to this are possible, and can use, for example, sensitization of other retinal cells such as the bipolar, horizontal and amacrine cell layers using a range of biological or genetic modification.

The light producing means may comprise an array of light sources each arranged to direct light along one of the light paths. The light sources may be LEDs or they may take other forms such as lasers, for example vertical cavity lasers. Alternatively the light path followed by light from a single light source, or a plurality of light sources, can be made variable to enable selection of light paths along which light will be directed at any particular time.

The control means may be arranged to simulate processing normally performed in the retina such that if the stimulating array of beams is incident on photosensitive cells, for example photo-sensitised ganglion cells of the retina, a sensation of the visual image corresponding to the captured image will be generated in the brain. Where stimulation of photo-sensitised retinal cells other than RGCs is intended, processing by the control means is simplified. The closer the photo-sensitised cell type is to the photoreceptor cell end of the photoreceptor to RGC signal transfer pathway the greater the proportion of image data processing carried out by it and subsequent cells in the cascade pathway.

Preferably the control means are arranged to perform an algorithm for selecting the group of light paths and the individual light beam time dependent intensities, and the algorithm can be updated to modify the relationship between the captured image and the selected group of light paths.

The present invention further provides a method of operating a retinal prosthetic device comprising capturing an image, defining a plurality of light paths along each of which a light beam can be directed towards a respective position on a retina, processing the captured image and produce a stimulating array of light beams along a group of the light paths, the group being dependent upon the captured image.

The present invention further provides a method of stimulating light sensitive cells in a retina comprising capturing an image, defining a plurality of light paths along each of which a light beam can be directed towards a respective position on a retina, processing the captured image and produce a stimulating array of light beams along a group of the light paths, the group being dependent upon the captured image.

Preferred embodiments of the present invention will now be described by way of example only with reference to the accompanying drawings

### BRIEF DESCRIPTION OF THE DRAWINGS

FIG. 1 is a schematic side view of a prosthetic device according to an embodiment of the present invention;

FIG. 2 is a schematic section through the device of FIG. 1;

FIG. 3 is a functional diagram of a control system of the device of FIG. 1;

FIG. 4 is a schematic diagram of an image processing part of the system of FIG. 3;

FIG. 5 is a functional diagram of a controller for an LED array of the device of FIG. 1; and

FIG. 6 is a diagram of the LED array of the device of FIG. 1.

### DESCRIPTION OF THE PREFERRED EMBODIMENTS

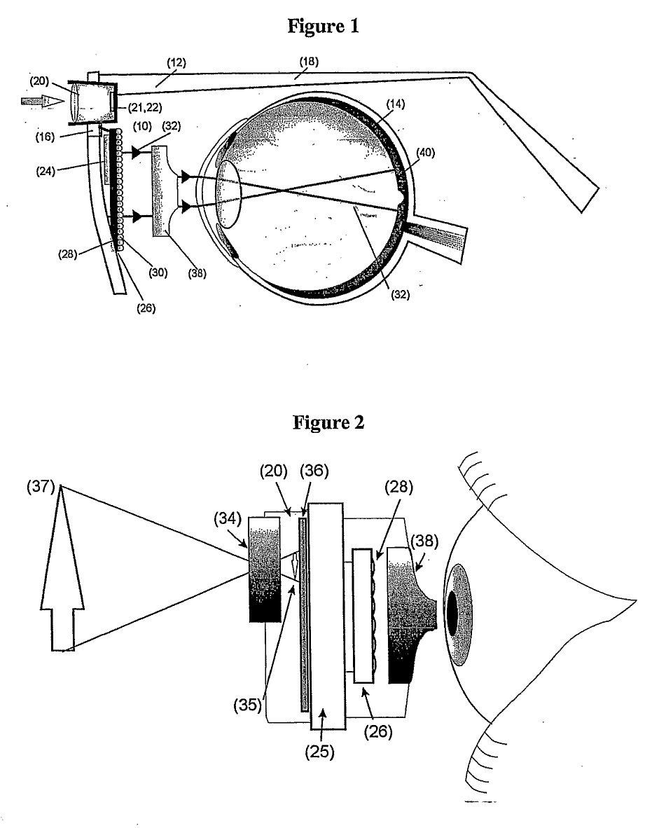

Referring to FIG. 1 a visual aid according to an embodiment of the invention comprises a retinal prosthetic device 10 mounted on a frame 12 which is arranged to support the device in front of a human eye 14. The frame is shaped in a similar manner to the frame of a pair of glasses and includes a bridge 16 arranged to rest on the bridge of the patient's nose and arms 18 arranged to be supported on the patient's ears. The prosthetic device 10 comprises in image capture system which includes a CMOS camera 20 and a processor 22 arranged to perform image processing functions. The device further comprises an LED stimulation addressing chip 24 and an array 26 of light sources in the form of LED devices 28 each of which can be turned on and off independently by the addressing chip 24. A lens 30 is located in front of each LED device 28 to focus the light that it emits into a focussed beam 32.

Referring to FIG. 2, which shows the prosthetic device 10 in a more schematic manner, the camera 20 includes a lens 34 arranged to focus an image 35 of an object or scene 37 onto the CMOS sensor array 21 part of the chip 36 of the camera 20. A printed circuit board 25 connects the output of the LED drivers 24 on the chip 36 with the LEDs 28. A further optical system 38 comprising appropriate lenses is arranged to shape the paths of the beams emitted from the LED devices 28 so that they are brought together and then extend out from the device, parallel to each other, over an area which is less than the area of the LED array 26. As shown in FIG. 1, this allows the light beams 32 to be directed into the eye 14, with each beam 32 being projected onto a respective point on the retina 40. The beams can be focussed so that each of them covers an area on the retina of diameter 50 to 200 micrometers. In this embodiment the number of light sources is limited only by the size of the LEDs and the size of the available space, whereas the power is not the limiting factor in principle, because it is supplied externally.

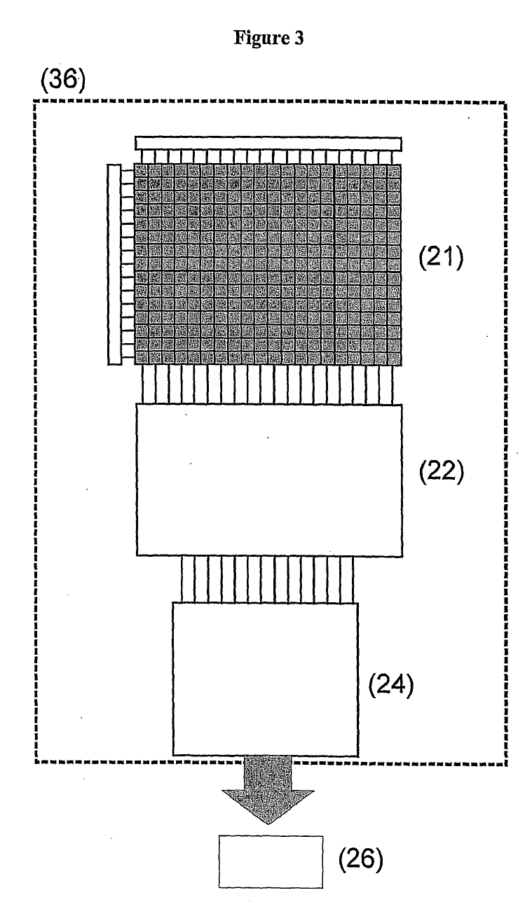

FIG. 3 shows the CMOS array 21, processor 22 and LED driver 24 in schematic manner. It will be appreciated that while the processor 22 is shown as one unit and the LED driver 24 as another, the functions of the two could be combined on a single chip or performed separately by a larger number of chips as appropriate.

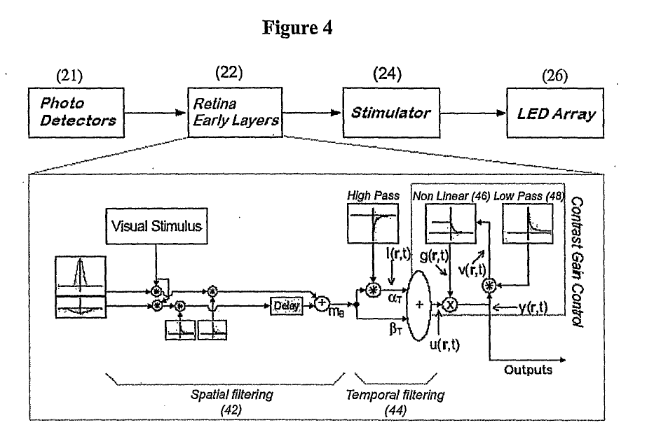

Referring to FIG. 4, the processor 22 is arranged to perform a number of operations on the signals from the CMOS array 21. As described above these simulate some of the processing functions normally carried out by the retina early layers. The processing in a normal retina converts the stimulation of the normal photoreceptors, rods and cones, as they detect light, to the stimulation of ganglion cells. The relationship between the photoreceptor stimulation and ganglion cell stimulation is complex. The ganglion cells that are stimulated depend partly on the positions of the stimulated photoreceptors on the retina, partly on the relationship between those positions, which in turn relates to shapes making up the viewed image, and partly on changes in those positions as the image being viewed changes, i.e. on movement within the viewed image. The processor 22 is therefore arranged to define for each of the LEDs (and hence the ganglion cell or cells that each LED will activate) a receptive field within the captured image. This is all areas of the captured image which will affect the control of that LED, and resembles as closely as possible the areas of the normal photoreceptor array of the eye that can influence firing of those ganglion cells.

The processor 22 is arranged to carry out spatial filtering of the viewed image in a spatial filter 42, in this case using a ‘difference of gaussians’ method which performs a type of edge detection. It is also arranged to carry out temporal filtering with a temporal filter 44 which is arranged to amplify the high temporal frequency components of the image to aid motion detection. Finally it performs contrast gain control using a low pass filter 46 and a non-linear filter 48 which modify the contrast in the image. Specifically this is arranged to maximise dynamic range and dark sensitivity.

The processor then analyses the filtered image and identifies spatial parameters of the image such as the presence, location and orientation of specific shapes of areas and lines in the image and then analyse the temporally filtered image to identify temporal parameters such as the speed and direction of linear and rotational motion of features in the image. An algorithm is then used which uses as inputs these parameters derived from these image processing steps, as well as the basic image data of the filtered or unfiltered images, to determine which of the LEDs needs to be turned on to stimulate the appropriate ganglion cells to cause the patient to ‘see’ an image corresponding to the viewed image.

The algorithm works on the basis of the scanning speed of the LED array, i.e. the time interval between consecutive updates in which LEDs are on and which are off. For each period the viewed image is analysed and the group of one or more LEDs that needs to be turned on is determined. Data identifying this group of LEDs is then sent to the LED driver 24.

The LEDs that need to be switched on and the timing for switching them on and off will depend partly on which of the ganglion cells need to be stimulated at any one time, and partly on the nature of light sensitizing that has been carried out on the ganglion cells. In some methods, the ganglion cells will be activated for as long as light of a particular wavelength or range of wavelengths is directed onto them. For other forms of light sensitizing, the ganglion cell can be activated or ‘turned on’ by directing light of a first wavelength onto it, and de-activated or ‘turned off’ by directing light of a different wavelength onto it. Therefore, the processor algorithm is arranged to identify first which ganglion cells need to be activated, and then which LEDs need to be turned on and off, and at what times, to produce the desired ganglion cell activation.

In this embodiment, light sensitization of the neural cells is achieved through genetic engineering as described by Hankins et al. Cells are induced to stably produce a specific protein (melanopsin) through heterologous expression of the gene encoding the synthesis of this novel opsin-type molecule. This molecule is incorporated into the cell plasma membrane and the photoreceptive function depends on the presence of cis-isoform of retinaldehyde. Melanopsin binds at specific site a retinaldehyde molecule, which undergoes isomer type transformation upon absorbing photon and in turn activates the protein, which is coupled to a G-protein type cascade. For the in-vitro demonstration system described by Hankins et al a cell line was grown, but for the present invention similar results in patients can be achieved, for example, through viral transfection. However it will be appreciated that there are other possible ways of sensitising neural cells to light which can also be used.

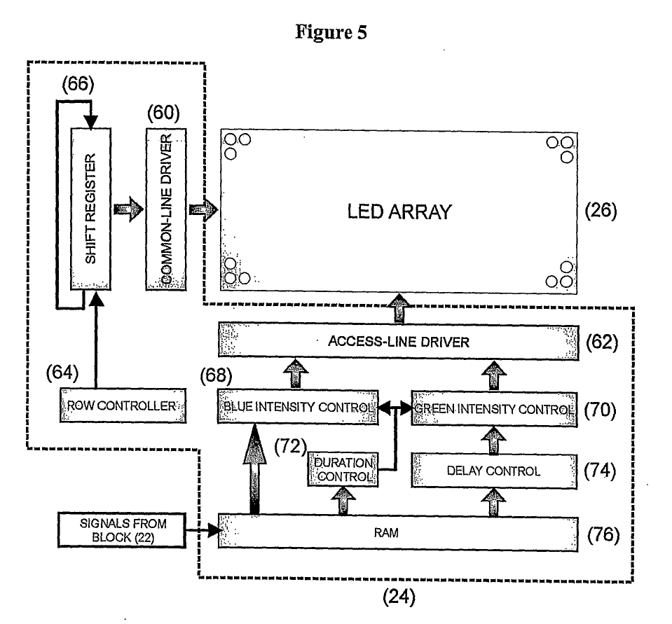

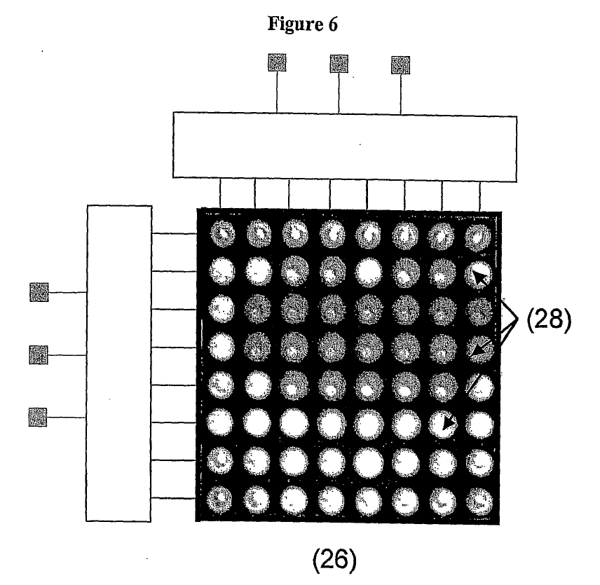

In this embodiment, the ganglion cells have been sensitized so that they are activated by blue light and they deactivate and recover through a series of spontaneously occurring processes. However, the LEDs used can also emit green light which could be used in some realisations of this proposal for deactivating the light sensitive molecules (so-called ‘push-pull’ mechanism) The LED array 26 is made up of rows of LEDs, each row comprising a number of pairs of blue and green LEDs, also shown in FIG. 6. Referring to FIG. 5, the LED driver 24 comprises a common line driver 60 which selects which row of LEDs within the array is to be active, and access line driver 62 which determines which LEDs within the active row are turned on and which turned off. The common line driver 60 is controlled by a row controller 64 via a shift register 66. The access line driver 60 is controlled by blue and green intensity control modules 68, 70 and duration control and delay control modules 72, 74. These modules process the data from the main processor 22, which is stored in RAM 76, to identify which LEDs need to be turned on and which turned off in each scanning period, and control the LEDs accordingly.

The main processor 22 and LED driver 24 are arranged so that their operation, including the algorithm used by the processor 22, can be modified. For example they can each be realised in Field Programmable Gate Array technology and hence can be externally tuneable. In this way feedback from the patient's experience can be relatively readily implemented by updating the algorithm.

It will be appreciated that the present invention has a number of advantages in comparison to known mainstream retinal implant proposals. The technique is non-invasive: the stimulating light beam should not damage cells, whereas stimulation electrodes are in intimate contact with the RGC membrane. The system gives flexible spatial control of the stimulation points. Conventional electrode implants once installed could not be moved around, but one can freely move light rays without damaging cells. The primate retina has a high degree of retinotopic distortion, so that RGCs are not mapped precisely to their corresponding input detectors, this is most apparent close to the macular region. With the embodiments described these spatial non-linearities may be neutralised. This advantage is especially important for the retinal implants, where a learning process combined with the spatial adjustment of the stimulating points is desirable for creating effective retinal implants. Since retinal implant can be used without major surgical interventions the cost will be considerably reduced compared to known methods using electrode implants. There is no requirement for power supply inside the eye. The whole device is external and thus larger external power supplies can be used. As the device is ex-vivo there will be no issues concerning sterility and infection.

1-16. (canceled)

17. A retinal prosthetic device for directing light beams onto respective positions on a retina, the device comprising a camera arranged to capture an image, a light source array and optical system arranged to define a plurality of light paths along each of which a light beam can be directed towards a respective one of the positions on the retina, and a controller arranged to process the captured image and control the light source array so as to produce a stimulating array of light beams along a group of the light paths, the group being dependent upon the captured image.

18. The device according to claim 17 wherein the light source array comprises an array of light sources each arranged to direct light along one of the light paths.

19. The device according to claim 17 wherein the optical system is arranged to shape the light paths so that they converge onto an area of the retina which is smaller than the area of the light source array.

20. The device according to claim 17 wherein the controller includes a spatial filter arranged to spatially filter the captured image.

21. The device according to claim 17 wherein the controller is arranged to identify at least one spatial parameter of the captured image and select the group of light paths in dependence thereon.

22. The device according to claim 17 wherein the controller includes a temporal filter arranged to temporally filter the captured image.

23. The device according to claim 17 wherein the controller is arranged to identify at least one temporal parameter of the captured image and select the group of light paths in dependence thereon.

24. The device according to claim 17 wherein the controller is arranged to simulate processing normally performed in the retina such that if the stimulating array of beams is incident on photosensitive cells of the retina a visual image corresponding to the captured image will be generated.

25. The device according to claim 24 wherein the cells are photo-sensitized retinal ganglion cells.

26. The device according to claim 17 wherein the controller is arranged to perform an algorithm for selecting the group of light paths, and the algorithm can be updated to modify the relationship between the captured image and the selected group of light paths.

27. A method of operating a retinal prosthetic device comprising capturing an image, defining a plurality of light paths along each of which a light beam can be directed towards a respective position on a retina, processing the captured image and producing a stimulating array of light beams along a group of the light paths, the group being dependent upon the captured image.

28. The method according to claim 27 wherein the prosthetic device is a device according to claim 17.

29. A method of stimulating light sensitive cells in a retina comprising capturing an image, defining a plurality of light paths along each of which a light beam can be directed towards a respective position on a retina, processing the captured image and producing a stimulating array of light beams along a group of the light paths, the group being dependent upon the captured image.

30. The method according to claim 29 wherein the light beams are directed at light-sensitized ganglion cells in a retina.
